(19) United States
(12) Patent Application Publication
(10) Pub. No.: US 2018/0353767 A1
(43) Pub. Date: Dec. 13, 2018

(54) AUXILIARY MAGNETIC STIMULATION (MS) COIL ARRANGEMENT AND SYSTEM

(71) Applicant: THE MAGSTIM COMPANY LIMITED, Whitland, Dyfed (GB)

(72) Inventor: Matthew Biginton, Whitland, Dyfed (GB)

(73) Assignee: THE MAGSTIM COMPANY LIMITED, Whitland, Dyfed (GB)

(21) Appl. No.: 15/780,709

(22) PCT Filed: Dec. 1, 2016
(86) PCT No.: PCT/GB2016/053775

§ 371 Date: Jun. 1, 2018

(30) Foreign Application Priority Data
Dec. 1, 2015 (GB) 1521133.7

(51) Int. Cl. A61N 2/00; A61N 2/02

(52) U.S. Cl. CPC A61N 2/006; A61N 2/02

(57) ABSTRACT

The present invention relates to an auxiliary Magnetic Stimulation (MS) coil arrangement and system, applicable particularly but not exclusively to Transcranial Magnetic Stimulation (TMS) arrangements. The invention provides an auxiliary MS coil arrangement for external magnetic stimulation of a body configured for cooperation with a primary MS coil winding. The auxiliary MS coil arrangement comprises an elongate conductive element formed of a current collecting portion for positioning in the magnetic field produced by a primary MS coil winding, a head portion comprising a magnetic stimulation winding having one or more turns, and a bridge portion extending between the current collecting portion and the head portion for enabling current flow from the current collecting portion to the head portion.

The present invention relates to an auxiliary magnetic stimulation (MS) coil arrangement and system, applicable particularly but not exclusively to Transcranial Magnetic Stimulation (TMS) arrangements.

Magnetic stimulating of neuromuscular tissue is well known and comprises a stimulating coil made up of one or more windings, each having a plurality of turns which may generate a succession of electrical discharge pulses producing magnetic pulses which further induce electrical signals in the tissue. Such a coil arrangement is disclosed in U.S. Pat. No. 6,179,770 where the magnetic stimulator generally comprises a charging circuit, a capacitor, a discharge control and a winding which is of a size and power rating appropriate for the generation of magnetic fields sufficient to cause stimulation of a body portion. The individual winding or plurality of windings each having one or more turns may be size adapted to fit partly over the cranium of a human patient in many applications, as well as being used for stimulation of other body parts. The winding(s) acts as an inductor and when connected to a stimulator which includes a capacitor provides an input voltage to the inductor which creates a circuit that passes an out of phase, sinusoidal voltage and current through the winding. An intense sinusoidal magnetic field is formed near the winding and is used to stimulate neurons or neuromuscular tissue in patients for medical and research applications. An example of a coil arrangement is a coil comprising a double winding coil that is generally in a plastic housing or a resin matrix that may suitably be positioned on a patient's cranium. The windings are made up of a single wound conductive element formed into a double winding configuration which is connected to the capacitor.

MS coils come in a variety of sizes, shapes and winding sizes for different stimulation applications. It is therefore common to have an array of different coil arrangements that may be utilised by the practitioner dependant on the different stimulation applications. The provision of a large number of coil arrangements is expensive. Furthermore, smaller coil arrangements are more complex to manufacture due to needing wire size of a minimum cross sectional area generally around 1mm wide to avoid issues of rapid overheating and requiring as many as fourteen turns on each winding to produce a double TMS coil of mean radius 25 mm. Therefore there is an inherent limit to the size of TMS coils can be made using conventional methods.

Aspects of the present invention provide a solution.

According to the present invention there is an auxiliary magnetic stimulation (MS) coil arrangement for magnetic stimulation of a body from external of the body configured for cooperation with a primary magnetic stimulation (MS) coil winding, the auxiliary MS coil arrangement comprising an elongate conductive element formed of:
a current collecting portion for positioning in the magnetic field produced by a primary MS coil winding;
a head portion comprising a magnetic stimulation winding having one or more turns; and
a bridge portion extending between the current collecting portion and the head portion for enabling current flow from the current collecting portion to the head portion.

It will be appreciated that the auxiliary coil arrangement is arranged to be for use external of a patient's body but on a patient's body. The auxiliary coil arrangement is preferably configurable to a desired position relative to a patient by a medical practitioner.

The head portion may be termed a secondary MS winding. Furthermore, the primary and preferably auxiliary MS coil arrangement are preferably Transcranial Magnetic Stimulation (TMS) coil arrangements.

A collected current is induced in the current collecting portion in the event the current collecting portion is positioned adjacent to and within the magnetic field produced by a primary MS coil winding. The collected current runs in the opposite direction to the current (in the primary MS coil) which created it. The collected current is transferred via the bridge portion to the head portion which effectively acts as a secondary magnetic stimulation coil. As such, the head portion effectively feeds off the current collecting portion to provide a magnetic stimulation winding that may be different in size and shape to the magnetic stimulation coil winding to which the auxiliary arrangement is positioned adjacent. The auxiliary (MS) coil arrangement acts in a manner similar to a transformer. However the primary coil may also still be used for stimulation, i.e. small and large stimulation zones may be achieved on a single MS coil.

Due to the differential in the turns of a primary magnetic stimulation coil winding and the preferably single or low number of turns in the current collecting portion, the voltage produced around the current collecting portion is reduced by the ratio of the number of turns in the primary winding to current collecting portion, whereas the current produced in the current collecting portion is increased in real terms (rather than theoretical) compared to the current in the primary MS coil winding by a factor of two to three times. The magnetic field produced in the head portion is proportional to both the current in the current collecting portion and the number of turns in the head portion. As a result of the increase in current in the current collecting portion it is possible to reduce the number of turns in the head portion and still achieve a desirable field output. This has the beneficial effect that less turns are needed in the auxiliary magnetic stimulation coil arrangement head portion thus enabling smaller coils to be made than by conventional manufacturing techniques. It is therefore generally desirable that the current collecting portion effectively comprises a single loop rather than a plurality of turns. However it is possible to achieve similar effects with multiple turns on the current collecting portion at the expense of more turns on the auxiliary head portion.

The elongate conductive element is beneficially a metal. The primary magnetic stimulation coil arrangement may be termed the primary coil and the head portion of the auxiliary (MS) coil arrangement may be termed the secondary or treatment coil.

It will be appreciated that the attachment arrangement is magnetically coupled by the current collecting portion to a primary magnetic stimulation coil arrangement. The current collecting portion is beneficially arranged to follow the contours of at least a portion of the primary magnetic stimulation coil winding. At least a portion of the current collecting portion is arranged to extend adjacent a peripheral edge of a magnetic stimulation coil winding. The peripheral edge is beneficially the radially outer edge of a magnetic stimulation coil winding. Preferably, the current collecting portion is substantially ring shaped however other shapes are possible. It is beneficial that the current collecting portion extends in use around a majority of the radially outer edge of a primary magnetic stimulation coil winding. It is also possible however that the primary MS coil winding may have other shapes such as elongated or ‘racetrack’ shapes for example.

The current collecting portion may comprise or may include a plate. The primary magnetic stimulation coil winding typically comprises a plurality of turns that lie in a generally common plane. The plate beneficially lies in a plane substantially parallel to the generally common plane of the turns of the winding of the magnetic stimulation coil winding.

The head portion may be substantially ring shaped. The head portion preferably follows the majority of the radially outer turn of a magnetic stimulation coil winding. In some embodiments the primary MS coil winding may be a different shape such as a “racetrack” configuration. It is beneficial that the shape of the current collecting portion generally follows this shape.

The current collecting portion preferably has a radially outer peripheral edge and the head portion has a radially outer peripheral edge, and the radially outer peripheral edge of the head portion is preferably but not necessarily shorter in length than the length of the radially outer peripheral edge of the current collecting portion for maximised current flow in the auxiliary coil. The area defined by the peripheral edge of the head is therefore beneficially less than the area defined by the peripheral edge of the current collecting portion.

The auxiliary (MS) coil arrangement beneficially comprises a temperature measurement arrangement for determining the temperature of the auxiliary magnetic stimulation winding.

The current collecting portion preferably comprises a radially outer edge and the magnetic stimulation attachment winding of the head portion comprises a radially outer edge, wherein the length of the radially outer edge of the head portion is preferably but not necessarily shorter in length of the radially outer edge of the current collecting portion.

The head portion may be detachably mountable relative to the current collecting portion. This enables changing over of the head portions depending on the required size parameters of the head portion for optimised and tailored treatment. Detachment may be possible in the bridge portion.

The head portion may comprise a plurality or turns. Preferably, the plurality of turns of the head portion is two or three for a single loop current collecting portion. However in the case of multiple turns for the current collecting portion more than three turns on the head portion may be required.

The current collecting portion may comprise a winding having a plurality of turns that may be nested. A single turn or looped portion is beneficial for the current collecting portion for optimising the reduction in number of turns in the head portion.

The auxiliary MS winding beneficially comprises an auxiliary housing.

It is beneficial in some embodiments to utilise to auxiliary magnetic stimulation (MS) coil arrangements in tandem. A first and second elongate conductive element may thus be provided and there may be an auxiliary housing having first and second auxiliary housing portions for receipt of the current collecting portions of the first and second elongated conductive elements respectively, the first and second auxiliary housing portions being spaced apart defining a slot therebetween for receipt of a primary magnetic stimulation (MS) coil winding. The slot may comprise a mouth to open to a void.

The primary MS winding can therefore be positioned between the current collecting portions of the first and second elongate conductive elements in a repeatable position ensuring repeatable treatment parameters in operation.

The first and second auxiliary housing portions are preferably in the form of prongs. The prongs beneficially extend from a third auxiliary housing portion which beneficially houses the bridge and head portions. It is further beneficial that the first and second housing portions are elongated. The void between the first and second housing portion is preferably defined by a substantially constant separation gap.

The auxiliary housing may be configured such that the head portion of a first and second auxiliary winding are moveable relative to each other to enable adjustment of their relative positions. For example in one embodiment the first and second head portions are stacked one above the other, whereas in an alternative embodiment they may be positioned in a side by side configuration.

The present invention extends to a magnetic stimulation (MS) coil system comprising a primary magnetic stimulation (MS) coil having at least one winding having one or more turns and an auxiliary magnetic stimulation (MS) coil arrangement as hereinbefore described, wherein the current collecting portion is positioned to cooperate with the primary MS coil winding such that a magnetic field produced by the primary MS coil winding induces a current in the current collecting portion.

It will be further understood with reference to the figures that one or more primary MS coil windings may be provided from which a current is induced in a current collecting portion of an auxiliary MS coil arrangement.

The auxiliary MS coil arrangement is preferably magnetically coupled to the primary MS coil winding.

A securing arrangement may be provided for securing the current collecting portion adjacent the winding. The securing arrangement may include one or more fixing elements arranged to secure the magnetic stimulation coil attachment arrangement adjacent the at least one winding. The current collecting portion is beneficially positioned adjacent a radially outer or a radially inner peripheral edge of the winding. The turns of the winding typically lie in a generally common plane and it is beneficial that the current collecting portion lies at least partially in that plane.

There is beneficially further provided a primary MS coil housing. The primary MS or coil housing is preferably elongate and is arranged to be grasped by a user. The housing for the primary MS coil arrangement therefore may effectively comprise a handle which may be manipulated by a user as appropriate to ensure that the magnetic field produced by the head portion is positioned in the correct location for treatment.

The primary MS coil may comprise a first winding and a second winding in a stacked configuration defining a space therebetween for receipt of the current collecting portion, the first and second windings configured to enable current flow in the same directions through the first and second windings respectively. The current collecting portion of the auxiliary MS coil arrangement may be positioned between the first and second windings. Due to the current flowing in the same direction through the first and second windings a magnetic field is produced by the first and second windings therebetween in opposing directions thus inducing a current in the current collecting portion.

The primary MS coil housing the first and second winding beneficially defines a slot therebetween for receiving the current collecting portion. The current collecting portion is therefore beneficially removably mounted between the first and second windings. The primary MS coil housing beneficially guides the current collecting portion into the space between a first and second winding. The position into which the current collecting portion is located is repeatable as providing a consistent induced current in the current collecting portion.

The auxiliary MS coil arrangement beneficially comprises first and second elongate conductive elements, wherein first and second current collecting portions of the first and second elongate conductive elements define a space therebetween for receipt of the primary MS coil winding. Accordingly, in this embodiment the first and second current collecting portions define a space therebetween into which a primary MS coil winding can be received.

The primary MS coil housing beneficially comprises a first and second slot for receipt of the first and second current collecting portions respectively.

An auxiliary housing beneficially comprises first and second auxiliary housing portions for receipt of the current collecting portions of the first and second elongate conductive elements respectively, the first and second auxiliary housing portions being spaced apart and stacked such that a void is defined between the first and second auxiliary housing portions and a mouth is open to the void for receipt of a primary MS coil winding. It is beneficial that a primary MS coil winding housing defines a first and second slot for receipt of the first and second auxiliary housing portions respectively.

The provision of the first and second auxiliary housing portions and corresponding first and second slots ensures repeatable positioning of the current collecting portions of the first and second windings with respect to the primary MS coil winding. Insertion is preferably limited by the provision of the third auxiliary housing portion which abuts against and beneficially secures to the primary MS coil winding housing.

The present invention will now be described by way of example only with reference to the accompanying drawings in which:

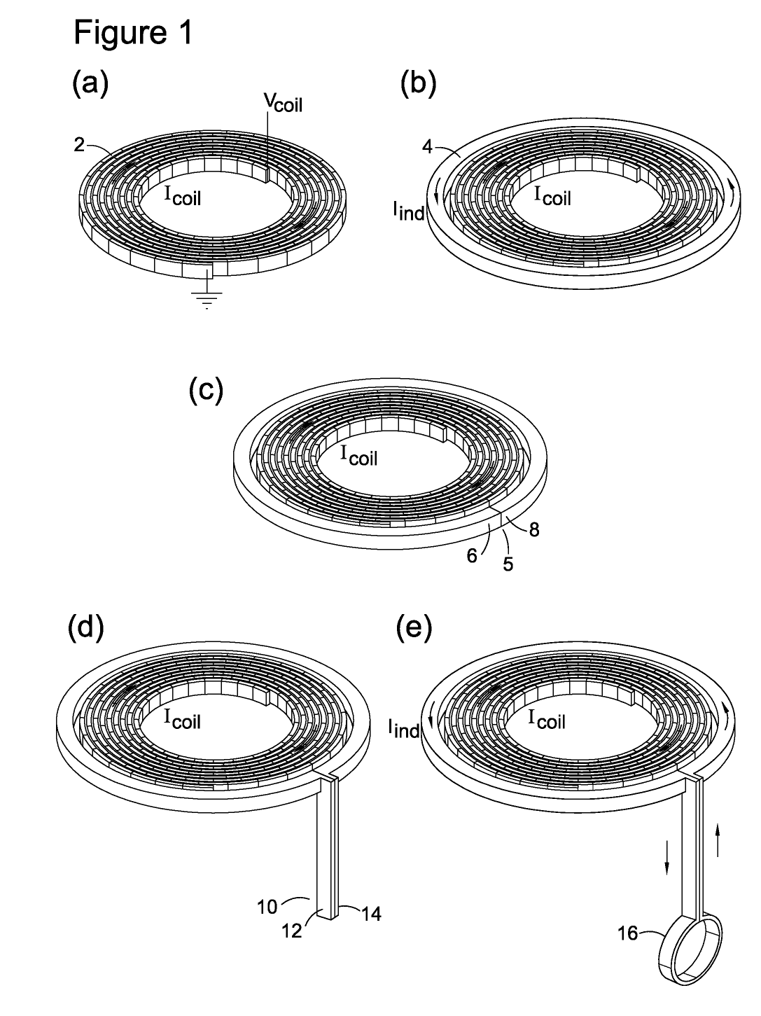

FIG. 1 is a schematic explanation of an auxiliary MS coil arrangement and system according to an illustrative embodiment where FIG. 1e is a schematic of an exemplary embodiment of the present invention.

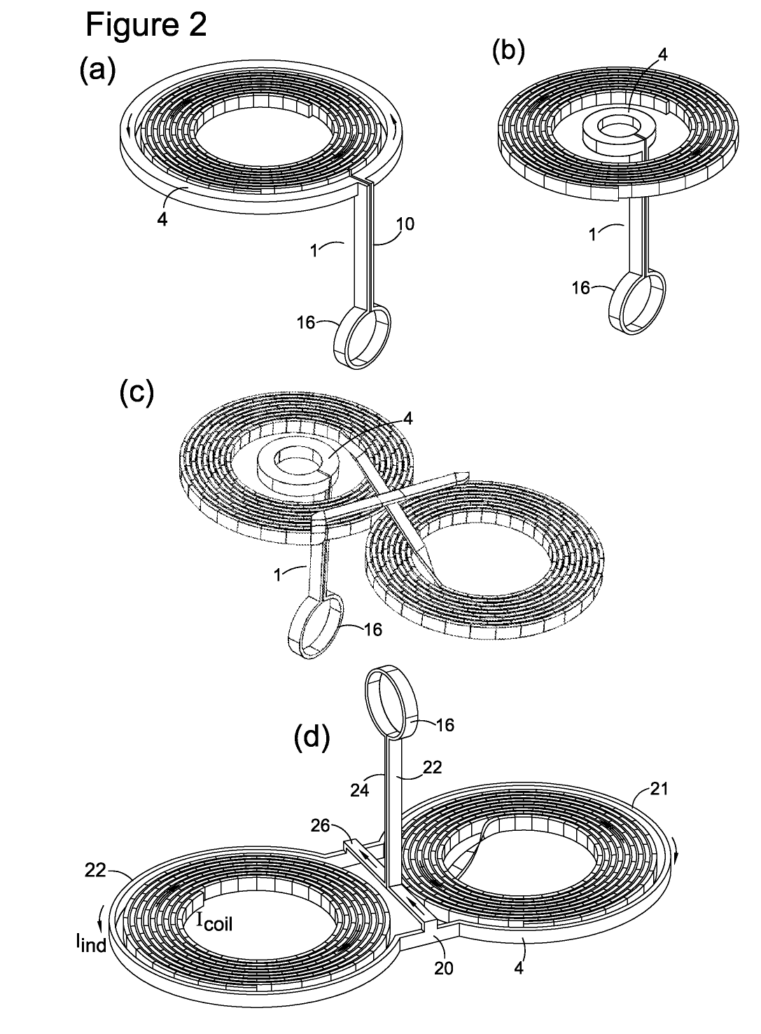

FIG. 2 is a presentation of illustrative embodiments of an auxiliary MS coil arrangement positioned adjacent a primary MS coil arrangement.

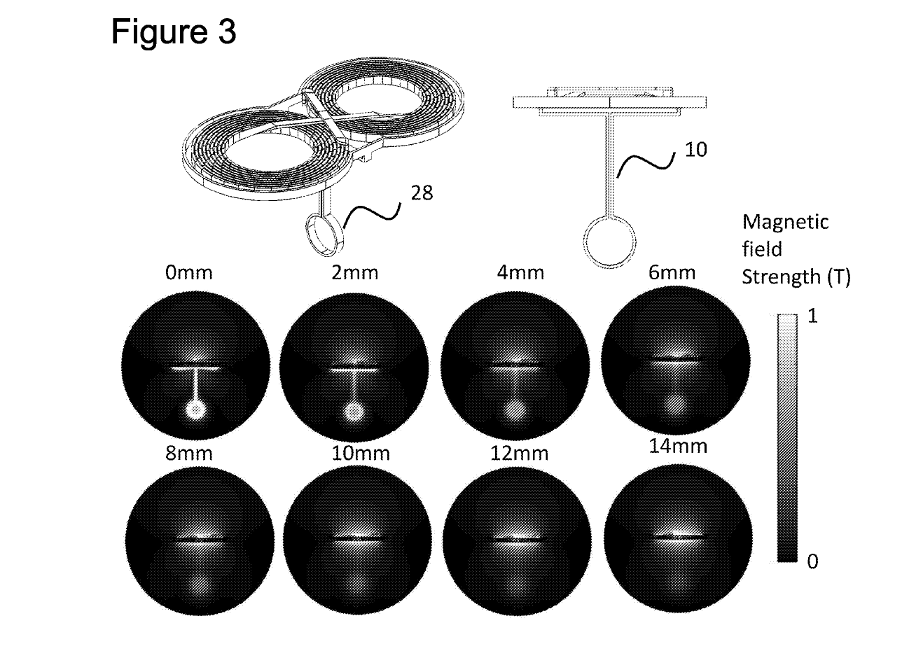

FIG. 3 is a schematic perspective and side view of an illustrative embodiment of the present invention adjacent to a primary MS coil and further shows peak magnetic field plots from finite element method of modelling presented as the distance from the head increases from zero millimetres to fourteen millimetres.

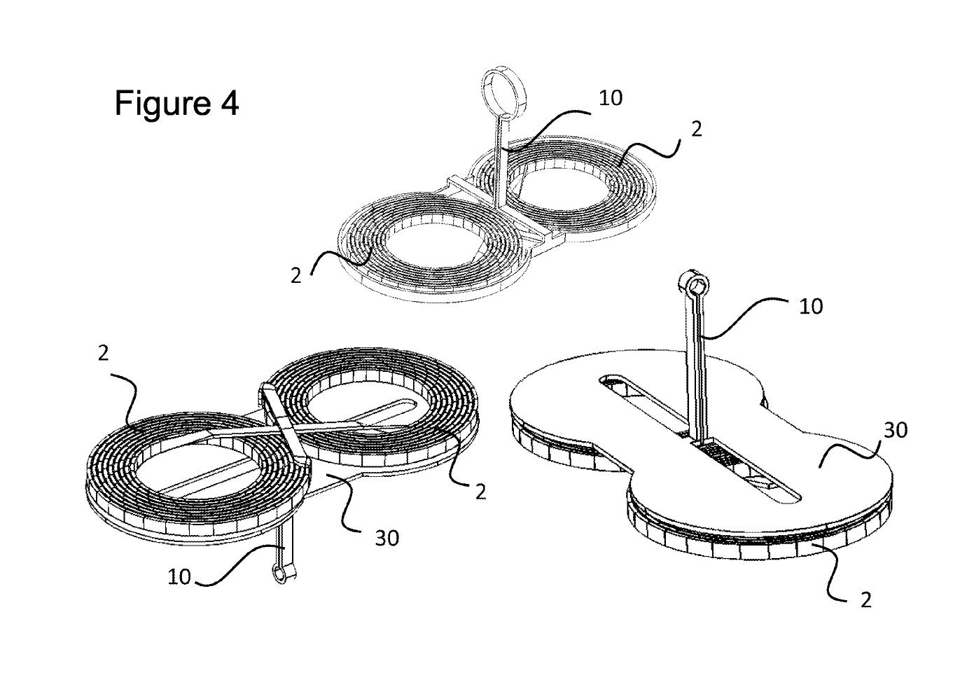

FIG. 4 is a schematic perspective view of a primary MS coil together with an auxiliary magnetic stimulation coil arrangement according to an illustrative embodiment of the present invention wherein the current collecting portion comprises a plate element.

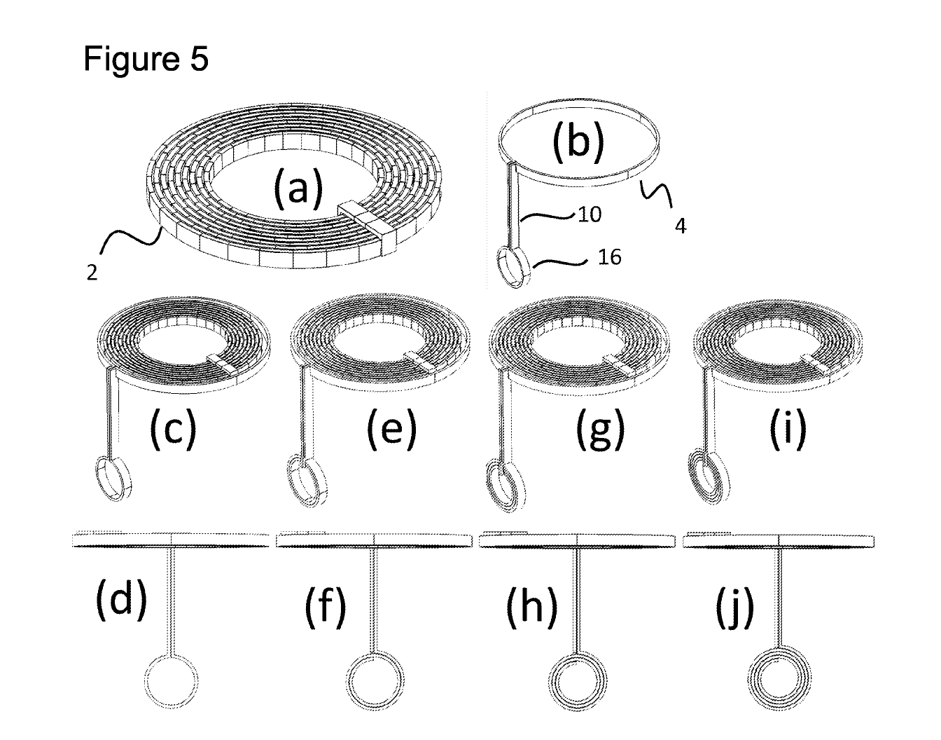

FIG. 5a and b show a primary MS coil and an auxiliary arrangement respectively according to an illustrative embodiment, and FIGS. 5c-i and d-j show perspective and side views respectively of four different auxiliary MS coil arrangements and primary coils, where the difference is in the number of turns of the treatment winding/head portion.

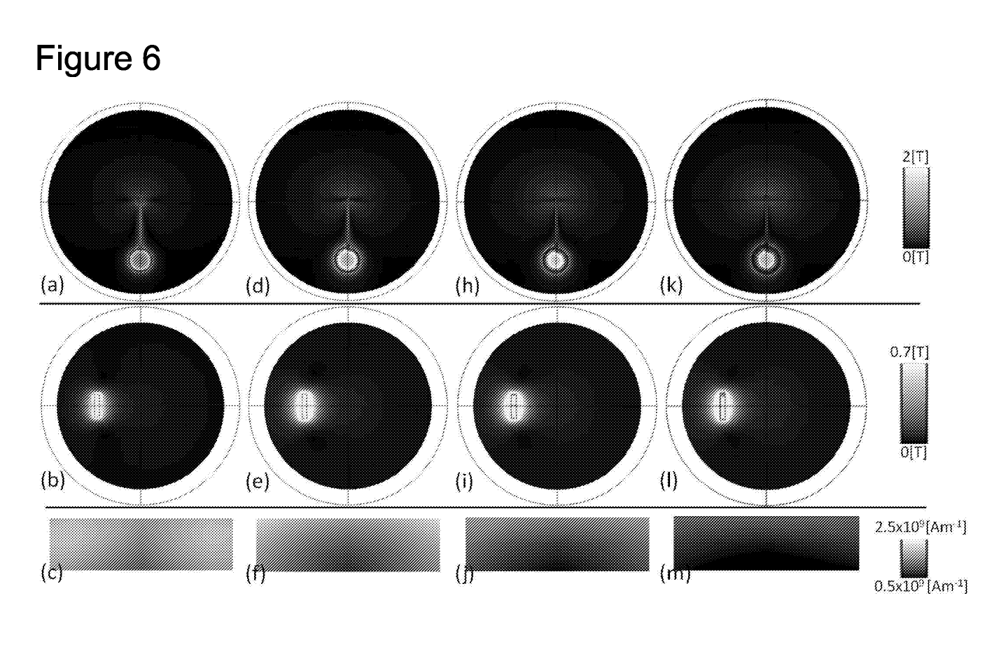

FIG. 6 is a presentation of the effects of the peak magnetic field plots from finite element method of modelling of the arrangements of FIG. 5, where FIGS. 6a, d, h and k are viewed as shown in FIGS. 5d, f, h and j respectively. FIGS. 6b, e, i and 1 show the same arrangement rotated about a vertical axis through 90 degrees. FIGS. 6 c, f, j and m show the current flow in the head portion.

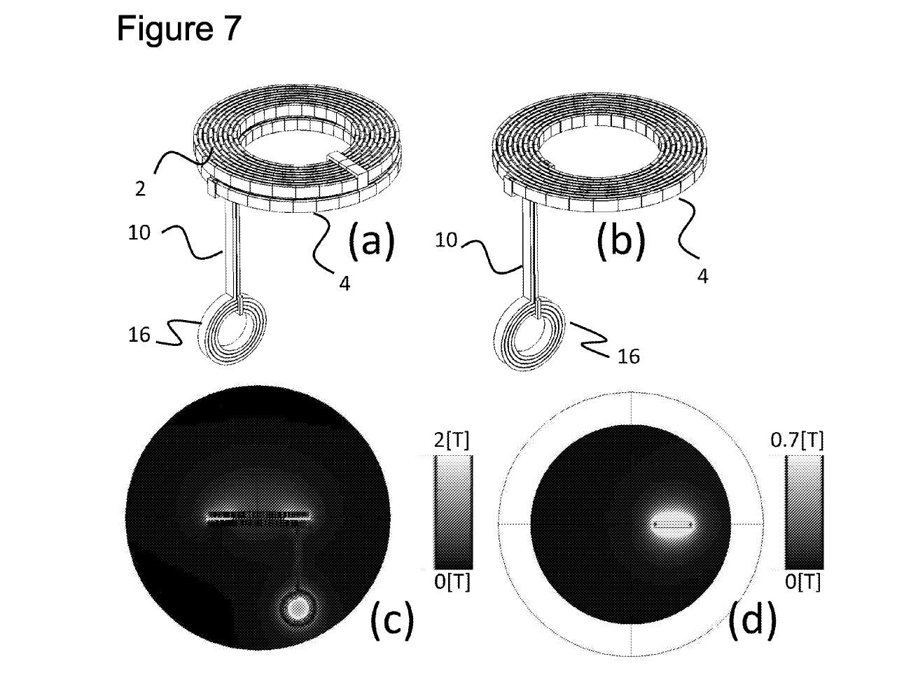

FIG. 7a is a schematic representation of an auxiliary MS coil arrangement according to an illustrative embodiment wherein the current collecting portion takes the form of a multiple turn winding adjacent to a primary MS coil winding. FIG. 7b shows the auxiliary MS coil arrangement alone, and FIGS. 7c and d show magnetic field plots for the head portion.

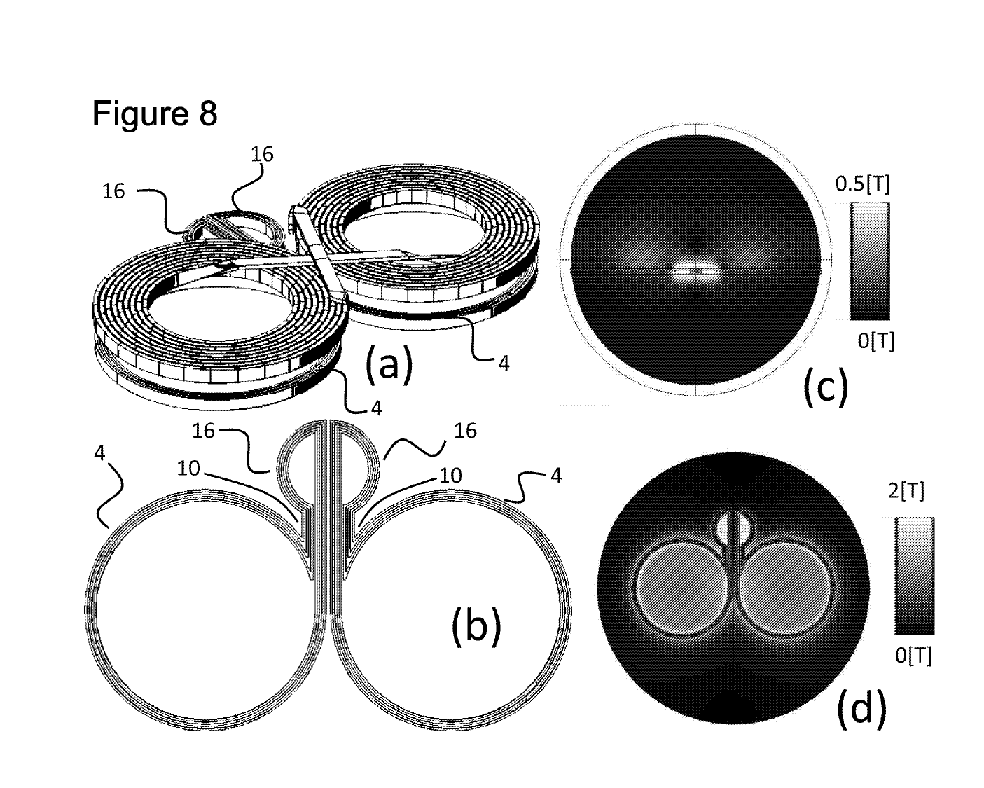

FIG. 8 is a schematic presentation of a further illustrative embodiment of the present invention, showing the use of further and second auxiliary MS coil arrangements.

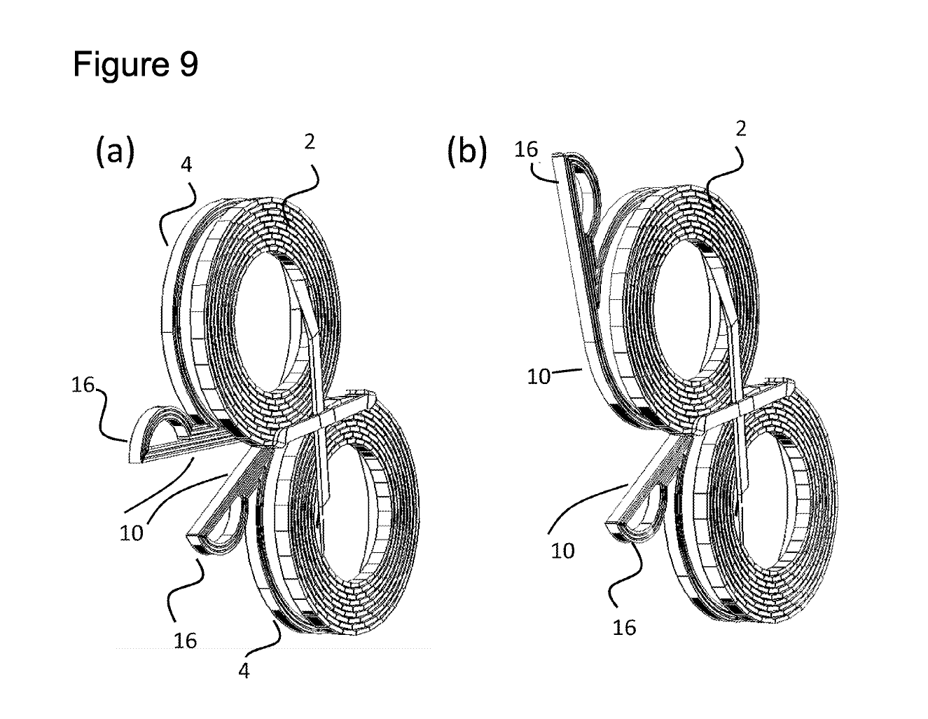

FIG. 9 is a schematic presentation of a further illustrative embodiment of the present invention.

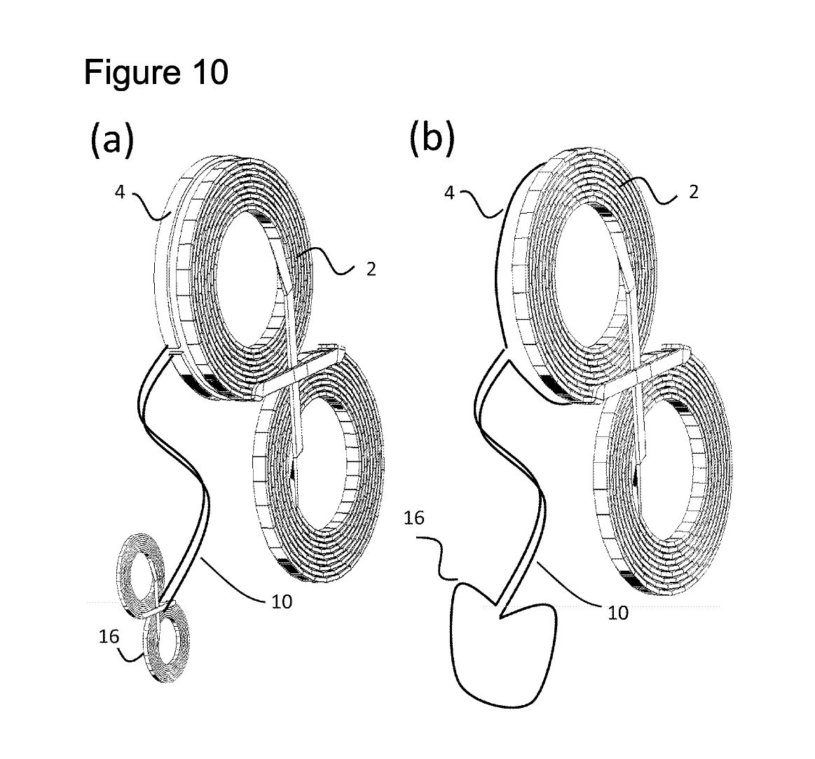

FIGS. 10a and 10b are further schematic presentations of illustrative embodiments of the present invention.

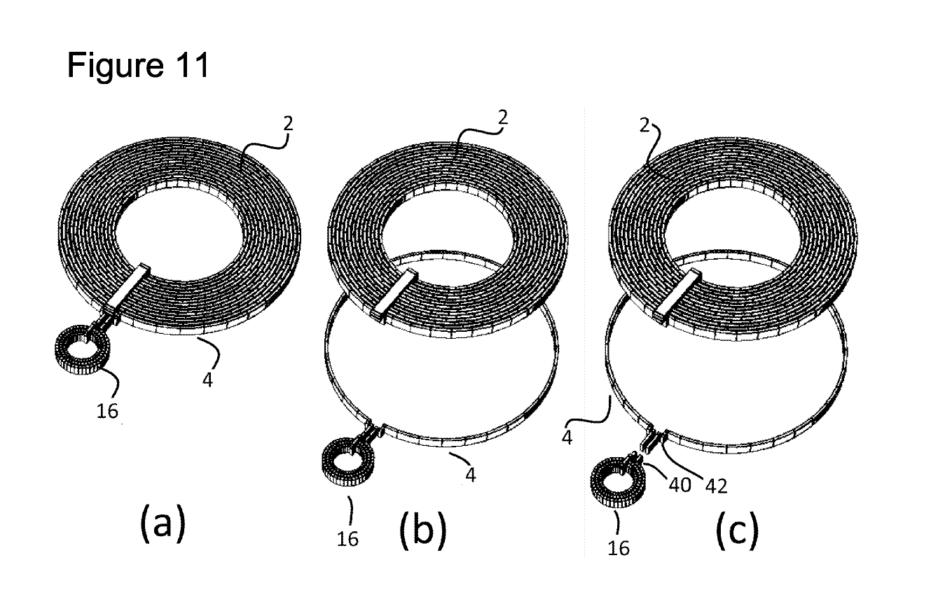

FIG. 11a-c show an illustrative embodiment of the present invention in position for use coupled to a primary MS coil, spaced apart from a primary MS coil, and wherein the head portion is separated from the current collecting portion.

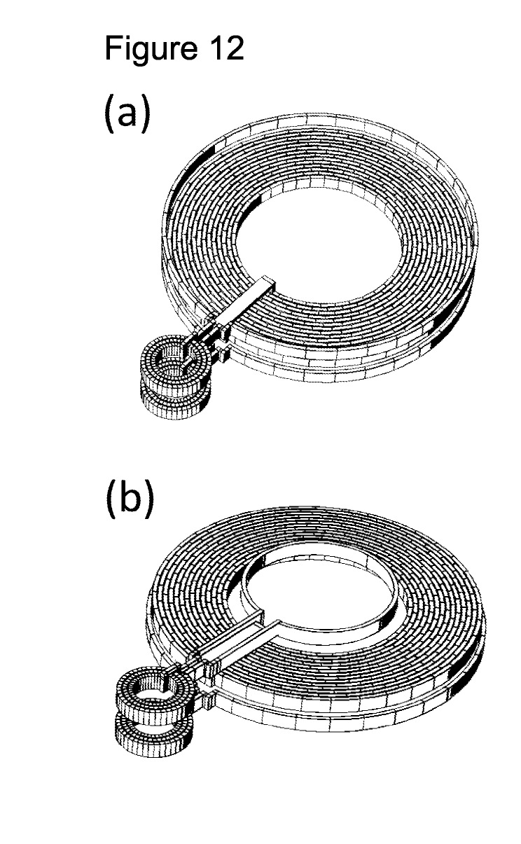

FIG. 12 is a schematic further illustrative embodiment of the present invention.

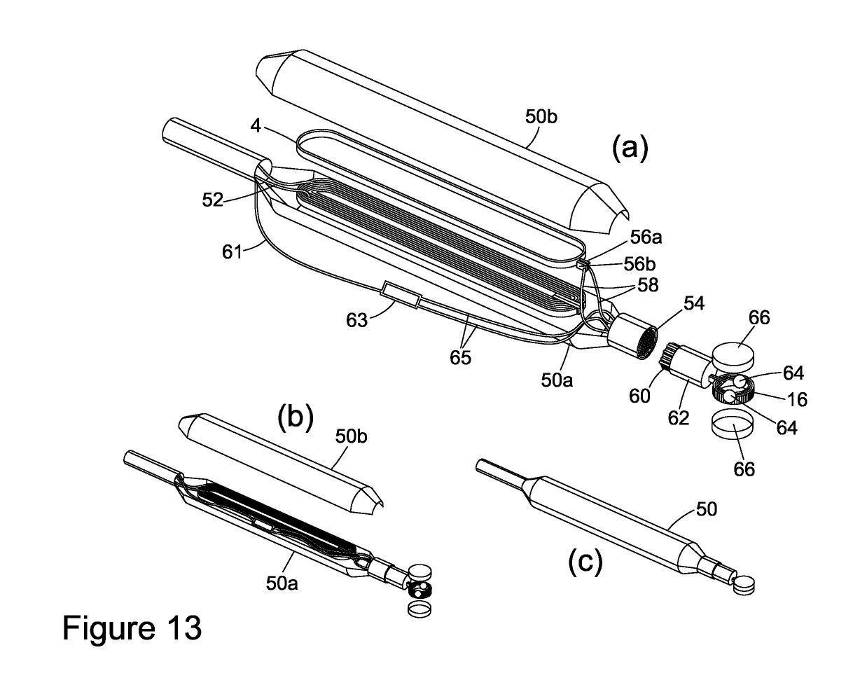

FIG. 13a-c is a schematic exploded, partially exploded and assembled illustrative embodiment of the present invention.

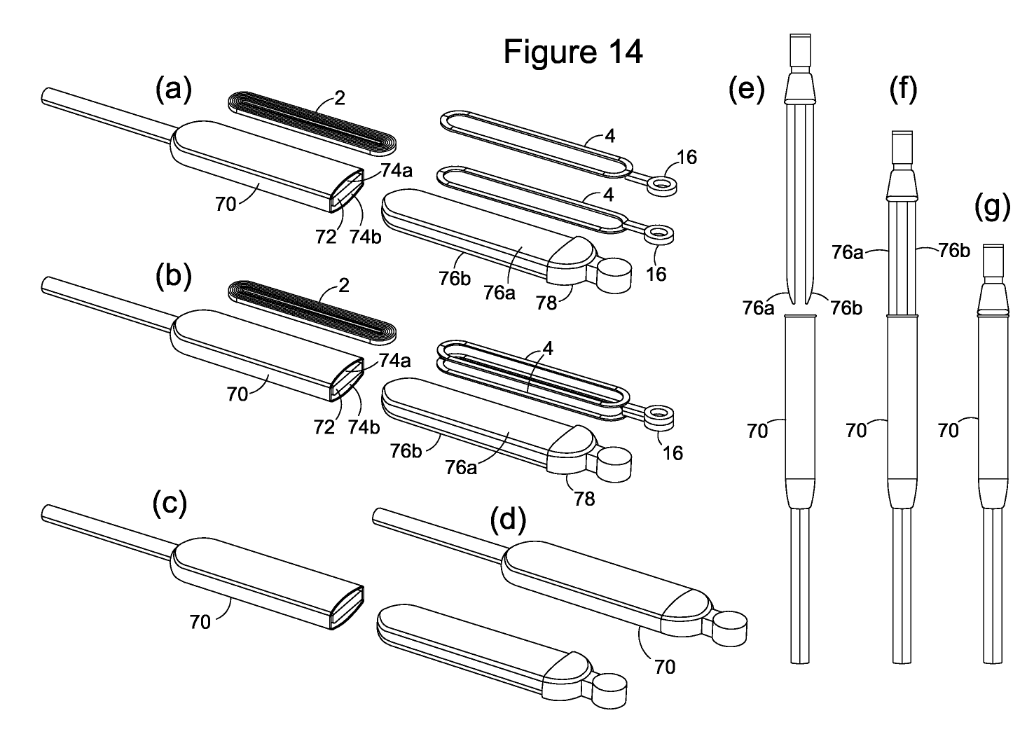

FIG. 14a-g is a schematic representation of an illustrative embodiment of the present invention in various stages of assembly from exploded view in FIG. 14a to completely assembled view in FIG. 14d and g.

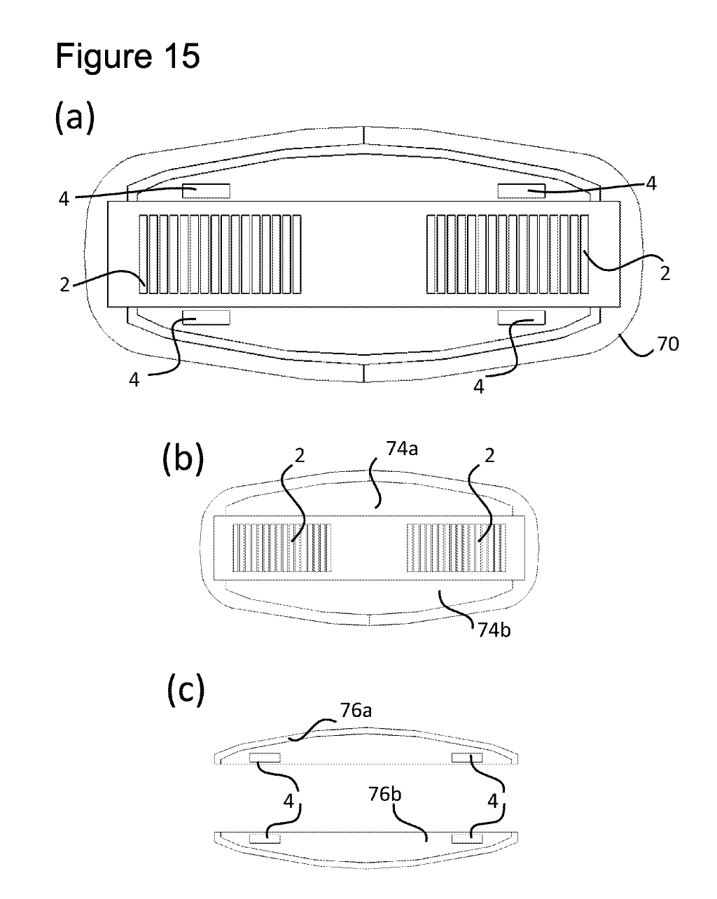

FIG. 15 is a schematic representation of a cross sectional view of FIGS. 14e to g respectively.

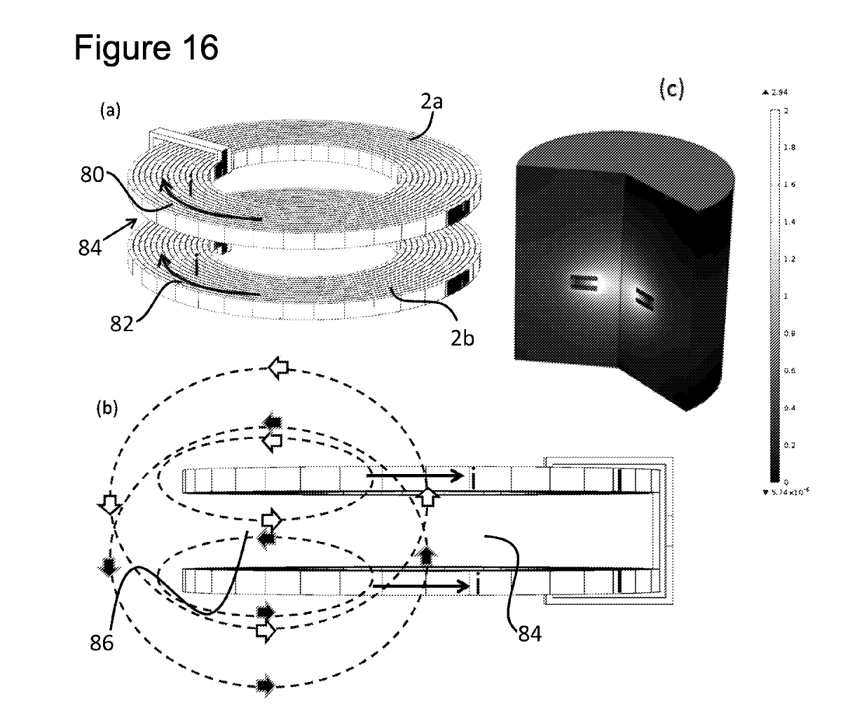

FIGS. 16a and b show a further illustrative embodiment of the present invention, and FIG. 16c shows an associated magnetic field plot for the primary MS coil windings.

Referring now to FIG. 1, a single magnetic stimulation coil (the primary coil) is presented in FIG. 1a showing a single winding 2 which generally has between one and many turns of metal forming a loop. A voltage (v_{coil}) is applied across the two ends of this winding which is generally provided by a capacitor inside a magnetic stimulator which is in series with the winding (an inductor). This forms a resonant LCR circuit and a sinusoidal current i_{coil} that is out of phase with the voltage and passes through the winding creating an intense magnetic field used to stimulate neurons and neuromuscular tissue in patients. If a solid continuous loop 4 (FIG. 1b) of wire is placed around the outside of the winding, the time variant magnetic field induces a current in this loop of wire which may be termed the current collecting loop 1_{IND}. This current runs in the opposite direction to the current which created it i_{coil}. If a slit 5 is placed in the loop as in FIG. 1c the current i_{IND} will cease in the loop and instead a potential difference will be formed between the ends 6, 8 which are now considered as terminals which can attach some extension wires or neck 10 two as in FIG. 1d. There is now a potential difference between terminals 12 and 14 at the bottom of the extension wires 10. These extension wires 10 could be any shape or length and be flexible or non-flexible and may point in any direction relative to the winding 2. This provides two new input wires for any head comprising one or more turns. For simplicity, shown is a simple single loop which acts as a new MS coil feeding off the primary winding via the current collecting portion 4. In adding the secondary loop a closed loop is formed between the current collecting portion and the extension terminals and the secondary MS winding and thus current will now flow around the current collecting portion and the secondary winding when the alternating current flows through the primary MS coil.

As shown in FIG. 1 it will be appreciated that the current collecting portion 4 is positioned adjacent the primary magnetic stimulation coil winding 2. It generally follows the contours of at least a portion of the magnetic stimulation coil winding and extends adjacent the peripheral edge of the primary magnetic coil winding 2. The current collecting portion 4 may be ring shaped and thus shown extends adjacent a radially outer edge of the magnetic stimulation coil winding.

Referring now to FIG. 2 there are a number of alternative ways of implementing the present invention. FIG. 2a is representation of the arrangement shown in FIG. 1 whereas in FIG. 2b the current collecting portion 4 is located radially inwardly of the primary magnetic stimulation coil winding 2, effectively nested within the primary winding. Alternatively the current collecting portion could be positioned both radially inwardly and radially outwardly of the primary magnetic stimulation coil winding. Additionally, the current collecting portion 4 may be positioned above or below the primary coil winding. In FIG. 2c there is presented a double winding configuration typically used in transcranial magnetic stimulation comprising a single elongate conductive element wound into two adjacent windings. The magnetic stimulation coil attachment arrangement generally designated with reference 1 is shown with the current collecting portion 4 radially inwardly of one of the windings.

Referring to FIG. 2c, a more complex embodiment of the invention is presented whereby the current induced from the current collecting portion 4 which extends around the majority of the peripheral edge of the double winding meets at the point identified by reference 20 as indicated by arrows 21, 22. The current flows up the bridge portion 10 comprising first and second extension wires 22, 24 and flows around the head portion 16, down the extension wire 24 and separates at point 26. It then begins flowing around the current collecting portion 4.

Referring now to FIG. 3, there is presented peak magnetic field plots from finite element method modelling of the coil attachment arrangement as presented in FIG. 2d. It is observed that there is an intense magnetic field generated by the head portion 16 as a result of the current running through it and this is as a product of magnetic coupling to the double coil windings. As presented in FIG. 3, the magnetic field produced and associated magnetic field strength reduces as would be expected as the distance indicated by arrow 28 increases. The magnetic field plots are shown varying between zero millimetres and fourteen millimetres. The magnetic field plots are presented in the same orientation as viewing the magnetic stimulation coil attachment arrangement 1 inside view as shown in FIG. 3.

Referring now to FIG. 4 the current collecting portion 2 is presented in the form of a plate in communication with the bridge portion 10. In the embodiment FIG. 4 the plate 30 is positioned below or above the windings 2. Utilising such a plate 30 has an advantage of reducing the magnetic field transferred from the magnetic stimulation coil winding to the patient. The amount of current collected by the plate 30 is dictated by the proximity of the plate 30 to the magnetic stimulation coil winding. This is the same for any configuration of current collection portions. As such the closer the current collection portion is to the magnetic stimulation coil winding the more current is generated in the current collecting portion, whether this be a loop of an elongate conductive element or a plate as presented in FIG. 4.

Referring to FIGS. 5 and 6, the current collecting portion 4 has one turn. The head portion 16 has one turn, as shown in FIG. 5c, increasing by one turn in FIG. 5e, to three turns in 5g and to four turns in 5i respectively. As shown in the magnetic field outputs as presented in FIG. 6, increasing the number of turns of the head portion 16 from one to two increases the magnetic field output, as does increasing the number of turns from two to three. However, increasing the number of turns to four has a reduced magnetic field output, due to the current reducing with more turns as shown in plots of current density in a cross-section of the current collecting loop in FIGS. 6(c), 6(f), 6(j) and 6(m) for one to four turns on the head portion respectively.

FIG. 7a is a schematic representation of an auxiliary MS coil arrangement wherein the current collecting portion 4 is in the form of multiple turns adjacent to a primary MS coil winding 2. FIG. 7b shown the auxiliary MS coil arrangement alone, and FIGS. 7c and d show magnetic field plots for the head portion 16, where the effect of the magnetic field produced by the head portion 16 can be clearly seen.

Referring to FIG. 8, two individual auxiliary MS coil arrangements are utilised to create the magnetic field as presented in FIGS. 8c and d. Such a configuration is beneficial particularly when it is difficult to appropriately position a current collecting portion 4 sufficiently close to the primary MS coil.

Referring to FIG. 9, a similar arrangement is presented as with respect to FIG. 8. It is beneficial in some applications that different parts of the brain are stimulated simultaneously. This is difficult to achieve with a single head portion, and as such utilising more than one auxiliary arrangements means first and second head portions can simultaneously treat different areas. There is no requirement for complex electronics to control two separate stimulators. Instead a current is passed through the primary MS coil arrangement causing a magnetic field to be product simultaneously by the first and second head portions 16.

Referring to FIG. 10a, in this embodiment the auxiliary MS coil arrangement shows a flexible bridge portion 10. Additionally, as an example only, the head portion comprises a double coil. Also as an example only the current collecting portion is provided magnetically coupled to and adjacent to one of the two provided primary coil windings. It will be appreciated that different combinations can be utilised, for example the head portion 10 does not necessarily have double windings, and double windings may be utilised with a rigid bridge portion 10.

Referring to FIG. 10b, in this embodiment the auxiliary MS coil arrangement is shown as being entirely flexible, and could for example be multi-strand wire, litz wire, or bundles of individually insulated wire.

Referring to FIG. 1a an auxiliary MS coil arrangement is shown coupled to a primary MS coil winding, and in FIG. 11b is shown schematically spaced apart from the primary MS coil winding, and in FIG. 11c is shown whereby the head portion 16 is detached from the current collecting portion. This detachment, via a plug 42 and socket 40 connection (or vice versa) for example, enables selection of a preferred head portion dependent on the size of area to be treated, or size of patient for example. This plug and socket or a separate plug and socket may include temperature sensor wires for monitoring the temperature of the auxiliary coil arrangement.

Referring to FIG. 12, in the embodiments of (a) and (b) two auxiliary arrangements detachably connectable as shown in FIG. 11 are coupled to a single primary MS coil winding to produce an effect similar to that described with reference to FIG. 8. In FIG. 12a the two auxiliary arrangements are arranged so that the head portions 16 are stacked above one another and the current collecting portions are 4 stacked around the outer radial edge of the primary coil. In FIG. 12b the first auxiliary MS coil arrangement is placed so that the current collecting portion 4 is around the outer radial edge of the primary coil whereas the second auxiliary arrangement is placed so that the current collecting portion is adjacent the inner radial edge of the primary coil.

It will be appreciated that in both embodiments of FIG. 12a the bridge portion 10 may be extended or the individual bridge portions 10 may diverge from one another so that the first and second head portions 16a, 16b can be positioned in a side by side or other configuration.

In the embodiments presented, the auxiliary MS coil arrangement is beneficially secured relative to the primary magnetic stimulation coil winding 2. The primary magnetic stimulation coil winding or windings 2 preferably includes a housing 70 and the auxiliary MS coil arrangement 1 is preferably secured to this housing in order that the current collecting position is fixed relative to the coil winding 2. This is typically achieved through the provision of fixing elements extending through the centre of the magnetic stimulation coil winding (S) which fixes the auxiliary MS coil arrangement to the housing. However, the inductance of magnetic stimulation coil winding drops as a consequence of the securing arrangement and to mitigate this any fixing elements such as screws or bolts which pass through the centre of the primary magnetic stimulation winding that are used to affix the auxiliary MS coil arrangement(s) may be made of ferromagnetic materials or have some ferromagnetic content for readjustment of the inductance of the auxiliary MS coil arrangement back to that of the primary magnetic stimulation coil winding.

It will also be appreciated that it is important in some embodiments to be able to measure the temperature of a winding to avoid overheating. This can be achieved with simple temperature probes connected to a circuit board housed in the housing for the primary coil winding(s). The auxiliary MS coil arrangement may include a temperature probe that is fed out of the auxiliary arrangement and into the housing for the primary magnetic stimulation coil winding. As such the current passing through the primary magnetic stimulation coil winding may be reduced or controlled in order to avoid overheating of the current collection portion.

Alternatively, commercially available wireless or radio frequency temperature sensors may be housed in the auxiliary MS coil arrangement (a transmitter) and in the primary magnetic stimulation winding housing to avoid the requirement for physical communication.

Referring to FIG. 13, in this embodiment the primary coil is substantially elongated in nature and resides in an elongated housing 50 which may be used as a handle comprising first and second housing portions 50a, 50b arranged to cooperate to house the primary MS coil winding and current collecting portion 4. At one end of the housing 50 resides the primary MS coil cable 52 and at the other end a pin connector socket 54 which may be a 48 way pin type. Around the periphery of the primary MS coil winding resides the current collecting portion 4. The two ends 56a, 56b of the current collecting portion 4 are connected to the 48 pin connector socket via flexible wires. FIG. 13a shows an exploded view for clarity and 13b shows the current collecting portion in its operational position.

A bundle of wires are shown 61 emerging from an end that connects to a stimulator to connect to a temperature sensor board 63 and two more bundles of wires 65 extend to the connector 54. The auxiliary coil head portion 16 is separated from the its current collecting portion 4 and is attached to a 48 way pin plug 60 along with two temperature sensors 64 that reside on the auxiliary coil. The auxiliary coil may be housed in a plastic casing 66. FIG. 13c shows the final assembly with the plug and socket connected to one another. In doing so the auxiliary coil head is electrically connected to the current collecting portion and the temperature sensor wires are electrically connected to the temperature sensor board.

Referring to FIG. 14 an embodiment of the present invention is presented wherein the primary coil winding 2 is elongated in nature into a so called “race track” configuration which is received in a primary housing 70. It will be appreciated that different shapes of primary coil winding may be utilised however, but elongation aids in providing a housing 70 capable of acting as a handle in use. The primary housing 70 houses the primary coil winding 2 within an insert 72. A first and second slot 74a, 74b are formed which are configured to receive first and second auxiliary housing portions 76a, 76b respectively. Housed within the auxiliary housing portion 76a, 76b are first and second current collecting portions 4 of first and second auxiliary MS coil arrangements. These slots 74a, 74b act to guide the auxiliary housing portions 76a, 76b such that the auxiliary MS coil arrangements are appropriately positioned. As shown in FIG. 14 it is beneficial that the auxiliary housing portion 76a, 76b are removably received in the corresponding slots 74a, 74b in order that different auxiliary MS coil arrangements may be utilised dependent upon the treatment effect required from the head portions 16. A third auxiliary housing portion 78 is provided which may house the bridge and head portions of the auxiliary MS coil arrangements. Again however different configurations may be achieved whereby the first and second auxiliary head portions are stacked one above each other, or are side by side, tilted relative to each other etc. It will be appreciated that the slot 74a, 74b define voids or cavities into which the auxiliary housing portion 76a, 76b can be positioned. Effectively therefore the current collection portions 4 of the first and second auxiliary MS coil arrangements are shown in FIG. 14 sit above and below the primary MS coil winding 2, and may not be in the plane of the primary MS coil winding, but may sit above and below this plane.

FIG. 14a shows a complete exploded view and FIG. 14b shows the relative positioning of the first and second auxiliary MS coil arrangements. FIG. 14c shows the components prior to assembly and FIG. 14d shows a dissembled configuration. FIGS. 14e, f and g show the same arrangement in side view clearly showing the first and second housing portions 76a, 76b separated from the primary MS coil winding 70 and subsequently being inserted and being completely inserted as shown in FIG. 14g.

Referring to FIG. 15a, cross sectional view of FIGS. 14, b and c are presented of the primary MS winding coil and housing and auxiliary coil and housing respectively.

Referring to FIG. 16 there is a schematic representation of FIGS. 16a and b and a further exemplary embodiment of the present invention. It will be appreciated that in the schematic representation the current collecting portion 4 of the auxiliary MS coil arrangement has not been included in the diagram to aid in clarity. In this embodiment first and second windings 2a, 2b are provided as shown in FIG. 16a in perspective view and in FIG. 16b in side view. The first and second windings 2a, 2b together form a single current flow path wherein as represented by arrows 80 and 82 enable current to flow through the turns of the first and second windings 2a, 2b in the same direction. A space 84 is defined between the first and second stacked windings 2a, 2b into which is received the current collecting portion 4 of an auxiliary MS coil arrangement. As indicated above, this has not been presented in the figures in order to aid in clarity.

As presented in FIG. 16b, the dashed lines represent the magnetic field produced by the first and second windings 2a, 2b respectively and the arrows represent the direction of the magnetic field. Above and below each of the windings 2a, 2b magnetic field lines are produced and the direction of each of these magnetic fields is anticlockwise as a result of the direction of current flow through the windings 2a, 2b. This means that in the location 86 in the space between the windings 2a, 2b the magnetic field is in opposite directions as is effectively underneath the first winding 2a and above the second winding 2b. The effect of an opposing magnetic field direction is to induce a current flow through a wire or in the present application a current collecting portion 4 positioned in this location.

As can be seen in conjunction with FIG. 16c, a cross section through the windings is represented and shows in the light area the intensified magnetic field where the arrows representative of field lines combine and in the darker area at location 86 where the field liens oppose and a current is induced.

A current collecting portion 4 in the form of a looped portion or plate portion having an opening therein is provided in this spacing and maximises the coupling of the current connecting portion to the first and second windings.

A housing is beneficially provided for housing the first and second windings 2a, 2b to define a slot therebetween for receipt of a current collecting portion to enable repeatable and consistent positioning of the current connecting portion relative to the windings.

The present invention has been described by way of example only and it will be appreciated to the skilled addressee that modifications and variations may be made without departing from the scope of protection afforded by the appended claims.

1. An auxiliary magnetic stimulation (MS) coil arrangement for magnetic stimulation of a body from external of a body configured for cooperation with a primary magnetic stimulation (MS) coil winding, the auxiliary MS coil arrangement comprising an elongate conductive element formed of:
a current collecting portion for positioning in a magnetic field produced by a primary MS coil winding;
a head portion comprising a magnetic stimulation winding having one or more turns; and
a bridge portion extending between the current collecting portion and the head portion for enabling current flow from the current collecting portion to the head portion.

2. An auxiliary magnetic stimulation (MS) coil arrangement according to claim 1 wherein the current collecting portion is arranged to follow the contours of at least a portion of a magnetic stimulation coil winding.

3. An auxiliary magnetic stimulation (MS) coil arrangement according to claim 1, wherein at least a portion of the current collecting portion is arranged to extend adjacent a peripheral edge of a magnetic stimulation coil winding.

4. An auxiliary magnetic stimulation (MS) coil arrangement according to claim 1, wherein the current collecting portion is substantially ring shaped.

5. An auxiliary magnetic stimulation (MS) coil arrangement according to claim 1, wherein the head portion is substantially ring shaped.

6. An auxiliary magnetic stimulation (MS) coil arrangement according to claim 1, wherein the bridge portion is elongate.

7. An auxiliary magnetic stimulation (MS) coil arrangement according to claim 1, wherein the current collecting portion has a length greater than the length of the head.

8. An auxiliary magnetic stimulation (MS) coil arrangement according to claim 1, wherein the current collecting portion has a radially outer peripheral edge and the head portion has a radially outer peripheral edge, and the radially outer peripheral edge of the head portion is shorter in length than the length of the radially outer peripheral edge of the current collecting portion.

9. An auxiliary magnetic stimulation (MS) coil arrangement according to claim 1, wherein a radius of the current collecting portion is greater than a radius of the head portion.

10. An auxiliary magnetic stimulation (MS) coil arrangement according to claim 1, wherein the current collecting portion comprises a radially outer edge and the head portion comprises a radially outer edge, wherein a length of the radially outer edge of the head portion is shorter in length than the length of the radially outer edge of the current collecting portion.

11. An auxiliary magnetic stimulation (MS) coil arrangement according to claim 1, comprising a temperature measurement arrangement for detecting the temperature of the magnetic stimulation attachment winding.

12. An auxiliary magnetic stimulation (MS) coil arrangement according to claim 1, wherein the head portion is detachably mountable relative to the current collecting portion.

13. An auxiliary magnetic stimulation (MS) coil arrangement according to claim 1, wherein the head portion comprises a plurality of turns.

14. An auxiliary magnetic stimulation (MS) coil arrangement according to claim 13 wherein the plurality of turns is two or three.

15. An auxiliary magnetic stimulation (MS) coil arrangement according to claim 1, wherein the current collecting portion comprises a winding having a plurality of nested turns.

16. An auxiliary magnetic stimulation (MS) coil arrangement according to claim 1, comprising a first and second elongate conductive element and further comprising an auxiliary housing having first and second auxiliary housing portions for receipt of the current collecting portions of the first and second elongate conductive elements respectively, the first and second auxiliary housing portions being spaced apart defining a slot therebetween for receipt of a primary MS coil winding.

17. An magnetic stimulation (MS) coil system comprising a primary magnetic stimulation (MS) coil having at least one winding having one or more turns and an auxiliary magnetic stimulation (MS) coil arrangement according to any preceding claim, wherein the current collecting portion is positioned to cooperate with the primary magnetic stimulation (MS) coil winding such that a magnetic field produced by the primary magnetic stimulation (MS) coil winding induces a current in the current collecting portion.

18. A magnetic stimulation (MS) coil system according to claim 17 wherein the auxiliary magnetic stimulation (MS) coil arrangement is magnetically coupled to the winding.

19. A magnetic stimulation (MS) coil system according to claim 17 further comprising a securing arrangement for securing the current collecting portion relative to the primary magnetic stimulation (MS) coil winding.

20. A magnetic stimulation (MS) coil system according to claim 19 wherein the securing arrangement includes one or more fixing elements arranged to secure the auxiliary magnetic stimulation (MS) coil arrangement relative to the primary magnetic stimulation (MS) coil winding.

21. A magnetic stimulation (MS) coil system according to claim 17 wherein the current collecting portion is positioned adjacent a radially outer or a radially inner peripheral edge of the winding.

22. A magnetic stimulation (MS) coil system according to claim 17 wherein the turns of the primary magnetic stimulation (MS) coil winding lie in a generally common plane, and the current collecting portion lies at least partially in that plane.

23. A magnetic stimulation (MS) coil system according to claim 17 wherein the current collecting portion is nested in the primary magnetic stimulation (MS) coil.

24. A magnetic stimulation (MS) coil system according to claim 17 comprising a primary MS coil housing, and preferably wherein the primary MS coil housing is elongate and is arranged to be grasped by a user.

25. A magnetic stimulation (MS) coil system according to claim 17 wherein the primary MS coil comprises a first winding and a second winding in a stacked configuration defining a space therebetween for receipt of the current collecting portion, the first and second windings configured to enable current flow in opposite directions through the first and second windings respectively.

26. A magnetic stimulation (MS) coil system according to claim 24 wherein the primary MS coil housing for housing the first and second winding define a slot therebetween for receiving the current collecting portion.

27. A magnetic stimulation (MS) coil system according to claim 17 wherein the auxiliary MS coil arrangement comprises first and second elongate conductive elements, wherein the first and second current collecting portions of the first and second elongate conductive elements define a space therebetween for receipt of the primary MS coil winding.

28. A magnetic stimulation (MS) coil system according to claim 24, wherein the primary MS coil housing comprises a first and second slot for receipt of the first and second current collecting portions respectively.

29. A magnetic stimulation (MS) coil arrangement or system according to claim 17 wherein the primary (MS) coil winding or the head portion of the auxiliary MS coil arrangement are at least partially surrounded or encapsulated in a ferromagnetic material.

30. (canceled)
